(19) United States
(12) Patent Application Publication
(10) Pub. No.: US 2012/0029243 A1
(43) Pub. Date: Feb. 2, 2012

(54) CATALYST- AND LIGNIN-COMPRISING COMPOSITION AND ITS USE FOR PREPARING AN AROMATICS COMPOSITION

(75) Inventors: Emmanouil PANTOUFLAS, Mannheim (DE); Roman Prochazka, Mannheim (DE); Stephan Schunk, Heidelberg-Rohrbach (DE)

(73) Assignee: BASF SE, Ludwigshafen (DE)

(21) Appl. No.: 13/192,797

(22) Filed: Jul. 28, 2011

Related U.S. Application Data
(60) Provisional application No. 61/368,666, filed on Jul. 29, 2010.

(51) Int. Cl. C07C 43/20; C07C 43/205; C07C 39/10; C10G 1/00; C07C 15/04; C07C 15/06; C07C 15/02; C07C 39/04; C07C 45/51

(52) U.S. Cl. 568/630; 568/716; 568/653; 568/651; 568/763; 568/806; 585/24; 585/240

(57) ABSTRACT

The present invention relates to a composition (“composite”) comprising lignin and at least one catalyst dispersed in the composition. The invention further provides a process for producing such a catalyst- and lignin-comprising composition and its use for preparing an aromatics composition.

The present invention relates to a composition (“composite”) comprising lignin and at least one catalyst dispersed in the composition. The invention further provides a process for producing such a catalyst- and lignin-comprising composition and its use for preparing an aromatics composition.

The large amounts of biomass which are continually produced in nature have hitherto been used to only a small extent as renewable raw material for utilization in terms of material or for energy generation. To conserve raw material resources, processes which make it possible to replace fossil raw materials by biomass starting materials are required. Here, very complete utilization of the biomass material provided is sought to achieve a high efficiency.

In processes for isolating cellulose, relatively large amounts of lignin are obtained in the form of black liquor as coproduct. In the classical pulping processes, e.g. the kraft digestion (sulfate digestion), this black liquor is generally utilized thermally in the paper mill and the remaining ash is worked up and reused in the digest process for the wood. New processes such as the LignoBoost process offer the opportunity of separating lignin from the black liquor and passing it to alternative uses. From a chemical point of view, lignin is interesting as a source of aromatics when it is possible to derive these in a high yield from the lignin obtained. Here, both the isolation of “monomeric” aromatics, i.e. aromatics having one benzene ring but different substitution patterns, and the isolation of “oligomeric” aromatics (which for the purposes of the present invention are compounds having up to 15 aromatic rings) or “polymeric” aromatics (which for the purposes of the present invention are compounds having more than 15 aromatic rings), i.e. aromatics in which a large number of aromatic rings which can have identical or different substitution patterns are bound to one another, are of interest. Aromatic compounds having a low molecular weight and specifically phenolic compounds have become widely used as intermediates and products of value. They serve, for example, as precursors for various resins, surface-active compounds, specialty chemicals, etc.

It is known that streams comprising lignin- or lignocellulose-comprising materials from various digestion processes can be subjected to an after-treatment to recover materials of value. An overview of the development of the catalytic pyrolysis of biomass starting materials may be found in G. Centi, R. van Santen, Catalysis for Renewables, From Feedstock to Energy Production, Wiley-VCH, Weinheim (2007), pages 119-147.

In Chem. Rev. 2006, 106, 4044-4098, G. W. Huber et al. describe the synthesis of fuels from biomass. According to that article, lignocellulose materials can in principle be converted into liquid fuels by means of three routes which differ in their primary step: gasification to synthesis gas, pyrolysis to biooil, hydrolysis to give sugars and lignin. The biooils obtained in the pyrolysis can subsequently be subjected to hydrodeoxygenation in the presence of hydrogen or to steam reforming.

U.S. Pat. No. 2,057,117 describes a process for preparing vanillin, in which a starting material selected from lignocellulose, a crude lignin extract and lignosulfonic acid is heated with an aqueous alkali metal hydroxide solution under superatmospheric pressure and the reaction mixture obtained is admixed with sulfuric acid in order to precipitate organic constituents and convert the vanillin into a soluble form.

WO 99/10450 describes a process for converting lignin into a hydrocarbon fuel. Here, lignin is subjected to a base-catalyzed depolymerization and subsequently hydroprocessing.

WO 2008/027699 A2 describes a process in which lignin originating from a pyrolysis of biomass is, after water-soluble constituents have been separated off, decarboxylated and hydrodeoxygenated and the organic products from this process step are subsequently subjected to hydrocracking.

WO 2010/026244 describes an integrated process for producing pulp and at least one low molecular weight material of value, in which
a) a lignocellulose-comprising starting material is provided and subjected to digestion with a treatment medium,
b) a cellulose-enriched fraction and at least one cellulose-depleted fraction are isolated from the digested material, where the cellulose-depleted fraction comprises at least part of the treatment medium from step a),
c) the cellulose-depleted fraction is subjected to a treatment to give at least one low molecular weight material of value and
d) the material/materials of value is/are isolated from the treatment product obtained in step c).

In an embodiment of this process, a cellulose-enriched fraction and a lignin-enriched fraction are isolated from the digested material, the lignin-enriched fraction is subjected to a depolymerization and an aromatics composition is isolated from the depolymerization product.

WO 2009/108601 describes a process for producing a starting material for biorefinery processes for producing a biofuel from a lignin-comprising starting material. Here, lignin from a black liquor of the pulping process or the black liquor itself is subjected to hydroprocessing in the presence of a hydrogen-comprising gas and a catalyst on an amorphous or crystalline oxidic support. Specifically, a heterogeneous molybdenum sulfide catalyst is used.

WO 2009/108599 has a disclosure content comparable to WO 2009/108601, with the focus on paper production.

U.S. 2009/0227823 describes a process for producing at least one liquid hydrocarbon product from a solid hydrocarbon starting material (e.g. a lignocellulose material), in which the starting material is subjected to catalytic pyrolysis and the pyrolysis products are subjected to a catalyzed after-reaction to give liquid products.

The unpublished European patent applications 10162255.3, 10162256.1 and 10162259.5 describe the preparation of organic materials of value from the digestion of lignocellulose-comprising starting materials.

In Angew. Chem. 2008, 120, 9340-9351, M. Stacker describes the catalytic conversion of lignocellulose-rich biomass to produce BTL (biomass-to-liquid) fuels in biorefineries. Here, pyrolysis of a lignin material obtained from the biomass to give biooil and a further work-up to give phenolic resins, synthesis gas, etc., are also shown schematically.

The term biooil generally refers to a liquid fraction from the pyrolysis of lignin or a lignin-comprising starting material, e.g. a lignocellulose material from wood and/or plant fibers. This is a composition comprising relatively high molecular weight organic compounds which are insoluble in water, mainly aromatic compounds (arenes). In present-day processes for the pyrolysis of lignin, a very high proportion of biooil as important intermediate in the production of biofuels and chemicals is generally sought.

It is an object of the present invention to provide an improved process for the pyrolytic preparation of an aromatics-comprising product mixture from a lignin-comprising starting material. The process should have at least one of the following properties:
1.) the total yield of aromatics based on the starting material should be as high as possible,
2.) the proportion of monomeric aromatics in the product mixture should be as high as possible,
3.) tar formation and/or buildup of adhering layers of carbonaceous material in the reactor space should be avoided as far as possible,
4.) the process should allow the inexpensive application of a catalytically active component to the starting material lignin.

A further object is to provide a catalyst-comprising composition which can advantageously be used in such a process.

It has surprisingly been found that the abovementioned disadvantages can be reduced or avoided when a lignin-comprising starting material is subjected to a pyrolysis, where the lignin-comprising starting material is partly or completely present as a composition (“composite”) which comprises at least one catalyst dispersed therein. It is particularly advantageous for the composite to be subjected to densification during contacting of the lignin-comprising starting material with the catalyst and/or subsequently thereto.

The invention firstly provides a composite comprising lignin and at least one pyrolysis catalyst distributed in the composite.

The invention further provides a composite which can be obtained by a process in which a lignin-comprising starting material is provided and is brought into intimate contact with at least one pyrolysis catalyst.

The invention further provides a process for producing a composite, wherein a lignin-comprising starting material is provided and is brought into intimate contact with a pyrolysis catalyst.

The invention further provides a process for preparing an aromatics composition from a lignin-comprising starting material, wherein a composite comprising lignin and at least one pyrolysis catalyst dispersed in the composite is subjected to pyrolysis.

In the abovementioned subjects, the material is preferably subjected to densification during and/or after contacting of the lignin-comprising starting material with the pyrolysis catalyst.

The invention further provides a process for preparing an aromatics composition from a lignin-comprising starting material, wherein:
a) a lignin-comprising starting material is provided,
b) the lignin-comprising starting material is brought into contact with a pyrolysis catalyst and subjected to compaction,
c) the compacted composite obtained in step b) is subjected to pyrolysis in a pyrolysis zone,
d) the discharge from the pyrolysis zone is subjected to separation to give the following three streams:
   D1) a solid catalyst-comprising fraction,
   D2) an aromatics-enriched fraction,
   D3) a fraction enriched in by-products which are more volatile than D2),
e) the fraction D1) obtained in step d) is at least partly recirculated to the pyrolysis zone.

The aromatics composition obtained by the process of the invention corresponds in terms of its composition to a high-quality biooil. It has a high proportion of low molecular weight and relatively high molecular weight organic compounds which are insoluble in water, mainly aromatic compounds. It is thus especially suitable as intermediate in the production of biofuels and chemicals.

The aromatics composition obtained by the process of the invention has a high proportion of aromatic compounds having up to 15 aromatic rings. The total content of aromatic compounds having up to 15 aromatic rings is, based on the total weight of the aromatics composition, at least 50% by weight.

The aromatics composition obtained by the process of the invention has a high content of single-ring aromatics. Single-ring aromatics are, for the purposes of the invention, also referred to as “monomeric aromatics”. Multiring aromatics having from 2 to 15 aromatic rings are also referred to as “oligomeric aromatics”. The aromatics composition obtained by the process of the invention preferably comprises at least 1% by weight, particularly preferably at least 2% by weight, of single-ring aromatics. The aromatics composition obtained by the process of the invention can advantageously be subjected to a subsequent dealkylation. The dealkylation products obtained in this way have a significantly increased content of single-ring dealkylated aromatics compared to the aromatics composition used.

The process of the invention makes it possible, in particular, to prepare an aromatics composition comprising monomeric aromatics selected from benzene, toluene, xylenes, ethylbenzene, phenol, phenol ethers, cresols, xylenes, guaiacols, veratroles, resorcinols, catechols, hydroquinones and further compounds different therefrom.

For the purposes of the invention, pyrolysis is a thermal treatment of the lignin-comprising starting material, with molecular oxygen not being introduced or introduced only in a small amount. Here, a small amount is an amount which is significantly smaller than the amount necessary for complete oxidation of the carbon comprised in the lignin-comprising starting material to CO₂. The amount of molecular oxygen introduced in the pyrolysis is preferably at least 50 mol % below, particularly preferably at least 75 mol % below, in particular at least 90 mol % below, the amount necessary for complete oxidation of the carbon comprised in the lignin-comprising starting material to Co₂. The pyrolysis generally occurs endothermically.

### Composite

For the purposes of the invention, a composite is a composition produced by bringing a lignin-comprising starting material and at least one pyrolysis catalyst into intimate contact with one another. The composite of the invention is comparable to a dispersion in which the catalyst is present as disperse phase finely dispersed in the lignin-comprising starting material as continuous phase. Lignin-comprising starting material and pyrolysis catalyst are preferably subjected to densification during and/or after contacting. Densifying is a physical treatment in which the components are subjected to a treatment under elevated pressure. Customary densification processes generally lead to the components also being brought into intimate contact. Densifying is associated with at least one of the following effects:
intimate contacting of lignin-comprising starting material and pyrolysis catalyst,
fine dispersion of the pyrolysis catalyst in the lignin-comprising starting material,
an increase in the density,
enlargement of the particle size,
optionally a reduction in the content of components which are liquid under normal conditions (20° C., 1013 mbar).

As regards suitable and preferred compacting apparatuses and compaction processes, reference is made to the information given further below. With regard to suitable and preferred lignin-comprising starting materials and pyrolysis catalysts, too, reference is made to the information given further below.

The composite of the invention preferably comprises the lignin in an amount of from 60 to 99% by weight, particularly preferably from 70 to 95% by weight, based on the total weight of the composite.

The composite of the invention preferably comprises the pyrolysis catalyst in an amount of from 0.1 to 20% by weight, particularly preferably from 0.2 to 5% by weight, based on the total weight of the composite.

The composite preferably has a content of components which are liquid under normal conditions (20° C., 1013 mbar) of not more than 20% by weight, particularly preferably not more than 5% by weight. In particular, the composite preferably has a water content of not more than 20% by weight, particularly preferably not more than 5% by weight.

The composite preferably has an average particle size in the range from 1 to 2000 μm, particularly preferably from 100 to 1000 μm.

The d90 of the composite is preferably not more than 2500 μm, particularly preferably not more than 2000 μm.

The d50 of the composite is preferably not more than 1500 μm, particularly preferably not more than 1250 μm.

The d10 of the composite is preferably not more than 600 μm, particularly preferably not more than 500 μm.

The abovementioned particle sizes can be determined by known methods, e.g. dynamic laser light scattering. To set a desired average particle size and a desired particle size distribution, it is possible to use the sieving devices customary for this purpose.

The composite preferably has a bulk density in the range from 0.3 to 0.8 g/l, particularly preferably from 0.4 to 0.7 g/l.

### Production of the Composite

The invention further provides a process for producing a composite comprising lignin and at least one pyrolysis catalyst dispersed in the composite. In this process, a lignin-comprising starting material is provided and brought into intimate contact with the pyrolysis catalyst. Densification is preferably carried out during and/or after contacting of the lignin-comprising starting material with the pyrolysis catalyst, i.e. contacting and densifying can be carried out separately at different times or partly or completely at the same time.

### Provision of a Lignin-Comprising Starting Material

The following statements in respect of lignin-comprising starting materials apply in their full scope both to the production of the composites of the invention and to the preparation of an aromatics composition by the inventive process described below.

Suitable lignin-comprising starting materials are pure lignin and lignin-comprising compositions. Here, the lignin content of the compositions is not critical within a wide range, although at excessively low lignin contents the process can no longer be carried out economically.

Preference is given to a lignin-comprising starting material comprising at least 10% by weight, preferably at least 15% by weight, based on the dry mass of the material, of lignin being provided. Preferred lignin-comprising compositions are compositions comprising from 10 to 100% by weight, particularly preferably from 15 to 95% by weight, based on the dry mass of the material, of lignin. For the purposes of the present invention, the term dry mass is as defined in the standard ISO 11465.

Lignocellulose-comprising materials are also suitable for providing a lignin-comprising starting material. Lignocellulose forms the structural skeleton of the cell walls of plants and comprises, as main constituents, lignin, hemicelluloses and cellulose. Further constituents of the cell walls of plants and therefore of lignocellulose-comprising materials obtained therefrom are, for example, silicates, extractable low molecular weight organic compounds (known as extractables, e.g. terpenes, resins, fats), polymers such as proteins, nucleic acids and plant gum (known as exsudate), etc.

Lignin is a biopolymer whose basic unit is essentially phenylpropane which, depending on the natural source, may be substituted by one or more methoxy groups on the phenyl rings and by a hydroxyl group on the propyl units. Typical structural units of the lignin are therefore p-hydroxyphenylpropane, guaiacylpropane and syringylpropane which are joined to one another by ether bonds and carbon-carbon bonds.

Suitable starting materials for the composites of the invention and the process of the invention for preparing an aromatics composition include both lignocellulose-comprising materials which are used with their natural composition without further chemical treatment, e.g. wood or straw, and lignin-comprising streams from the processing of lignocellulose, e.g. from processes for cellulose production (pulp processes).

The lignocellulose materials which can be used according to the invention can be obtained, for example, from wood fibers and plant fibers as starting material. Preferred lignocellulose materials are those from wood and residues of the wood-processing industry. They include, for example, the various types of wood, e.g. wood from broadleaved trees such as maple, birch, pear tree, oak, alder, ash, eucalyptus, common beech, spruce, yew, hemlock, pine, larch, fur, cedar, etc. Wood can be divided not only into wood from broadleaved trees and that from conifers but also into “hardwoods” and “softwoods”, which are not synonymous with the terms broadleaved tree timbers and conifer timbers. In contrast to hardwood, the term softwood refers to lighter wood (i.e. wood having an oven-dry density below 0.55 g/cm³, for example willows, poplars, limes and virtually all conifers). All hardwoods and all softwoods are in principle suitable for producing the composites of the invention and for use in the process of the invention for preparing an aromarics composition. The wood used in the process of the invention can also be present in manufactured form, e.g. in the form of pellets. Suitable residues from the wood-processing industry are not only scrap wood but also sawdust, parquetry grinding dust, etc. Further suitable lignocellulose materials are natural fibers such as flax, hemp, sisal, jute, straw, coconut fibers, switchgrass (Panicum virgatum) and other natural fibers. Suitable lignocellulose materials are also obtained as residues in agriculture, e.g. in the harvesting of cereal (wheat straw, maize straw, etc.), maize, sugarcane (Bagasse), etc. Suitable lignocellulose materials are also obtained as residue in forestry, e.g. in the form of branches, bark, wood chips, etc. Another good source of lignocellulose materials is short rotation crops which allow high biomass production on a relatively small area.

A lignin-comprising stream from the digestion of a lignocellulose material for producing cellulose (pulp) is preferably used for providing the lignin-comprising starting material. The digestion makes possible an at least partial separation of the lignocellulose-comprising starting material into cellulose and materials accompanying cellulose, with lignin being among the latter.

In a preferred embodiment, a lignocellulose-comprising material is provided, subjected to digestion and a cellulose-enriched fraction and a lignin-enriched (and at the same time cellulose-depleted) fraction are isolated from the digested material.

Processes for digesting lignocellulose-comprising materials to produce cellulose are known in principle. Lignin-comprising streams from all digestion processes known to those skilled in the art are suitable for use in the process of the invention. These processes can basically be divided, on the basis of the treatment medium used, into aqueous-alkaline processes, aqueous-acidic processes and organic processes. An overview of these processes and the digestion conditions may be found, for example, in WO 2010/026244.

The treatment medium used for digesting the lignocellulose-comprising materials is capable of solubilizing at least part of the lignin. The cellulose comprised in the lignocellulose-comprising material, on the other hand, is generally not solubilized or solubilized to only a minor extent in the treatment medium. A cellulose-enriched fraction is then preferably separated off by filtration or centrifugation.

A lignin-comprising (cellulose-depleted) fraction which comprises, in addition to lignin, at least one further component selected from hemicellulose, cellulose, degradation products of the abovementioned components, digestion chemicals and mixtures thereof is preferably isolated from the digested material.

In many cases it is not critical to the production of the composites of the invention and the pyrolysis for preparing an aromatics composition if a lignin-comprising starting material comprising at least one further component in addition to lignin is used.

If a lignin-comprising fraction which comprises at least one further component in addition to lignin is used for providing the lignin-comprising starting material, at least part of the compounds other than lignin can be removed before contacting with the pyrolysis catalyst. The components removed from the lignin-comprising fraction (organic components and/or inorganic process chemicals) are preferably passed to a further work-up and/or thermal utilization, preferably within the process for cellulose production from which the lignin-comprising fraction was obtained.

To remove at least part of the compounds other than lignin, the pH of the lignin-comprising fraction can firstly be adjusted to a suitable value. For adjusting the pH, lignin-comprising fractions from aqueous-alkaline processes (such as the kraft process) can be admixed with an acid. Suitable acids are, for example, CO₂ (or the carbonic acid resulting therefrom with water), mineral acids such as hydrochloric acid, sulfuric acid and phosphoric acid. Lignin-comprising fractions from aqueous-acidic processes can be admixed with a base to adjust the pH. Suitable bases are, for example, alkali metal bases such as sodium hydroxide or potassium hydroxide, alkali metal carbonates such as sodium carbonate or potassium carbonate, alkali metal hydrogencarbonates such as sodium hydrogencarbonate or potassium hydrogencarbonate and alkaline earth metal bases such as calcium hydroxide, calcium oxide, magnesium hydroxide or magnesium carbonate and also ammonia or amines.

The removal of at least part of the compounds other than lignin from the lignin-comprising fraction is preferably carried out by filtration, centrifugation, extraction, precipitation, distillation, stripping or a combination thereof. A person skilled in the art can control the composition of the lignin-comprising fraction and thus of the composite by the separation process. The at least partial removal of the components other than lignin can be carried out in one or more stages. Customary filtration processes are, for example, cake filtration and deep bed filtration (e.g. as described in A. Rushton, A. S. Ward, R. G. Holdich: Solid-Liquid Filtration and Separation Technology, VCH Verlagsgesellschaft, Weinheim 1996, pages 177 ff., K. J. Ives, in A. Rushton (editor): Mathematical Models and Design Methods in Solid-Liquid Separation, NATO ASI Series E No. 88, Martinus Nijhoff, Dordrecht 1985, pages 90 ff.) and crossflow filtrations (e.g. as described in J. Altmann, S. Ripperger, J. Membrane Sci. 124 (1997), pages 119-128). Customary centrifugation processes are described, for example, in G. Hultsch, H. Wilkesmann, “Filtering Centrifuges,” in D. B. Purchas, Solid-Liquid Separation, Upland Press, Croydon 1977, pages 493-559; and in H. Trawinski, Die äquivalente Klärfläche von Zentrifugen, Chem. Ztg. 83 (1959), pages 606-612. Extraction can be carried out using, for example, a solvent which is not miscible with the treatment medium from pulp production or at least one solvent which has a miscibility gap and in which lignin and optionally further desired components are soluble in a sufficient amount. The removal of components which can be vaporized without decomposition from the lignin-comprising fraction can be carried out by customary distillation processes known to those skilled in the art. Suitable apparatuses for the work-up by distillation comprise distillation columns such as tray columns equipped with bubble caps, sieve plates, sieve trays, ordered packing, random packing elements, valves, side offtakes, etc., evaporators such as thin film evaporators, falling film evaporators, forced circulation evaporators, Sambay evaporators, etc., and combinations thereof.

In a specific embodiment, a lignin-comprising stream from the digestion of a lignocellulose material which still comprises at least part of the liquid treatment medium from the digestion is used to provide the lignin-comprising starting material. The lignin-comprising stream is then preferably subjected to a precipitation of a lignin-comprising fraction followed by a partial or total removal of the liquid components in order to provide the lignin-comprising starting material for the production of the composite of the invention.

Preference is given to using a lignin-comprising stream from the digestion of a lignocellulose material by means of an alkaline treatment medium for providing the lignin-comprising starting material. Particular preference is given to using a black liquor, in particular a black liquor from sulfate digestion (kraft digestion). To provide a lignin-comprising material, a black liquor from the kraft digestion can firstly be acidified to precipitate at least part of the lignin comprised and the precipitated lignin can subsequently be isolated. The abovementioned acids are suitable for acidification. The pH of the black liquor is preferably reduced to a value of not more than 10.5. The isolation of the precipitated lignin is preferably carried out by means of a filtration process. Suitable filtration processes are those mentioned above. If desired, the isolated lignin can be subjected to at least one further work-up step. Such steps include, for example, a further purification, preferably washing with a suitable washing medium. Suitable washing media are, for example, mineral acids such as sulfuric acid, preferably in aqueous solution. The LignoBoost process is particularly suitable. LignoBoost processes are described in WO 2006/038863 (EP 1797236 A1) and WO 2006/031175 (EP 1794363 A1), which are hereby incorporated by reference.

In a specific embodiment, the lignin-comprising starting material is subjected to compaction before contacting with the pyrolysis catalyst. In the case of such a “precompaction”, too, further compaction is generally carried out during and/or after contacting with the pyrolysis catalyst.

### Pyrolysis Catalyst

For the purposes of the invention, the term “pyrolysis catalyst” also refers to catalyst precursors which are converted into the catalytically active form only before or during the pyrolysis.

Preference is given to using an acidic pyrolysis catalyst as pyrolysis catalyst. Suitable acidic pyrolysis catalysts are Lewis-acid and Bronsted-acid pyrolysis catalysts.

The pyrolysis catalysts which are suitable for use in the composites of the invention are preferably selected from:
I) water-insoluble catalysts
II) water-soluble catalysts and mixtures thereof.

Preferred water-insoluble catalysts I) are characterized by a defined acidity. Suitable catalysts I) are oxidic catalysts which are preferably selected from oxides, oxide mixtures or mixed oxides of the main group and transition elements.

Oxide mixtures are mixtures which comprise at least two oxides of the main group and transition elements.

Mixed oxides (also referred to as multimetal oxides) have at least two different metal atoms in addition to oxygen, where the metals can be different metals or the same metals in different oxidation states. Mixed oxides have an X-ray-crystallographically uniform phase.

The catalysts I) can be crystalline, partially crystalline or amorphous. Particularly preferred water-insoluble catalysts I) are oxides, oxide mixtures or mixed oxides of the elements magnesium, boron, silicon, aluminum, phosphorus, iron, nickel, cobalt, calcium, sodium and sulfur. Very particular preference is given to oxides, oxide mixtures or mixed oxides of the elements magnesium, boron, silicon, aluminum, phosphorus, iron and sulfur. Oxides, oxide mixtures or mixed oxides of the elements boron, silicon and aluminum are especially preferred. The oxides, oxide mixtures or mixed oxides can in each case be crystalline, partially crystalline or amorphous.

Further particularly preferred water-insoluble catalysts I) are solid-state compounds having acidic properties, known as solid-state acids. These include zeolites. Suitable zeolites are in principle the crystalline, naturally occurring or synthetic framework silicates known under this name. These can vary in terms of their composition, but generally have at least one alkali metal and/or alkaline earth metal in addition to silicon, aluminum and oxygen. In a specific embodiment, a naturally occurring zeolite is used.

Further suitable catalysts I) are zeolite-analogous structures, acidic aluminum oxides and modifications thereof. Preference is given to modifications which have an influence on the strength or number of the acidic sites. The invention therefore firstly provides a nickel-comprising catalyst which can be obtained by treatment of a zeolite with an acidic aqueous nickel salt solution and subsequent calcination.

To produce a modified zeolite, it is possible, for example, to subject a zeolite to modification with metal cations. This includes, for example, treatment with an acidic nickel salt solution. The treatment of the zeolite with the acidic aqueous metal salt solution is generally effected by intimate contacting of the zeolite with the solution. This results in ion exchange of cations of the zeolite (alkali metal/alkaline earth metal cations) with metal cations of the treatment solution. Contacting of the zeolite with the salt solution is preferably effected by customary dipping and impregnation processes as are known for catalyst production. The pH can be adjusted by addition of inorganic acids such as hydrochloric acid, nitric acid, sulfuric acid or phosphoric acid. For example, a nickel salt whose aqueous solution already has a suitable acidic pH is used for producing the modified zeolites. As nickel salts, preference is given to using nickel(II) salts. These are used, in particular, in the form of the nitrates, sulfates or acetates.

Further suitable water-insoluble catalysts I) are crystalline or partially crystalline or amorphous oxides, oxide mixtures or mixed oxides which are obained as by-product in a catalyst factory during crushing or sieving. Catalysts which are removed as deactivated catalysts in particular processes, for example the FCC process, are also suitable.

Preferred water-soluble catalysts II) have, in combination with the lignin-comprising starting material used for producing the composites of the invention, a particular acidity profile which is advantageous in the process of the invention.

Particularly preferred water-soluble catalysts II) are selected from compounds of groups 1, 2, 3 to 12, and 13 to 16 of the Periodic Table of the Elements and mixtures thereof. In particular, the water-soluble catalysts II) comprise at least one compound which comprises one of the following elements: Na, K, Mg, Ca, Sr, La, Ti, V, Cr, Mn, Fe, Co, Ni, Cu, Zn, B, Al, Si, P and S. The water-soluble catalysts II) very particularly preferably comprise at least one compound comprising one of the following elements: Na, Ca, La, V, Mn, Fe, B, Al, Si, P and S.

Suitable water-soluble catalysts II) are in principle covalent compounds, complexes and salts of the abovementioned elements. Suitable salts of these elements can have inorganic or organic counterions. Possible water-soluble catalysts II) are, in particular, compounds which are inexpensive and/or do not introduce undesirable compounds into the pyrolysis reaction, which components have to be removed by means of complicated work-up steps. Such undesirable components can be, for example, halogens such as chlorine or bromine or compounds thereof. Preference is given to compounds which do not have to be removed from the pyrolysis process or whose decomposition products do not have to be removed from the pyrolysis process. These include salts whose anions are preferably selected from sulfate, sulfide, sulfite, acetate, hydroxide, carbonate and nitrate. If a solvent is used for producing the composites of the invention, preference is given to catalysts which are readily soluble in the solvents used for producing the composites. If, in a specific embodiment of the production of the composites of the invention, a solvent other than water or a water-comprising or water-free solvent mixture is used, preference is given to catalysts which are soluble therein.

The pyrolysis catalysts used for producing the composites of the invention are preferably selected from boric acid, zeolites, silica, alumina, aluminosilicates, zirconium oxide, titanium dioxide, water-soluble nickel salts and water-soluble cobalt salts.

A specific pyrolysis catalyst is boric acid.

A further specific pyrolysis catalyst is a zeolite ZSM-5.

In a further specific embodiment, a naturally occurring zeolite is used as pyrolysis catalyst.

A further specific pyrolysis catalyst is boehmite as is marketed, for example, under the tradename Disperal by Sasol Deutschland GmbH.

A further specific pyrolysis catalyst is nickel acetate.

A further specific pyrolysis catalyst is cobalt acetate.

In a specific embodiment, the pyrolysis catalyst comprises the catalyst-comprising solid reactor discharge from a pyrolysis in the presence of a composite as defined above.

Contacting and compaction

The production of the composite of the invention comprises contacting of the lignin-comprising starting material with the pyrolysis catalyst (=step a)).

The production of the composite of the invention optionally further comprises joint densifying of the lignin-comprising starting material and the pyrolysis catalyst.

In a first preferred embodiment, step a) is carried out alone, i.e. no additional densification of the lignin-comprising starting material and the pyrolysis catalyst is carried out. Suitable apparatuses for carrying out only step a) are, for example, drum mixers, rotary tubes or other suitable mixers.

In a second particularly preferred embodiment, the lignin-comprising starting material and the pyrolysis catalyst are subjected to densification during and/or after contacting (steps a) and b)).

Densification is preferably carried out by compacting, extrusion, tabletting or a combination of at least two of these measures.

Steps a) and b) can be carried out at separate times or partly or completely together. In a preferred embodiment, the lignin-comprising starting material is firstly brought into contact with the pyrolysis catalyst under conditions under which essentially no densification occurs and the resulting mixture is subsequently densified. Densification is generally associated with intimate contacting of the components. Steps a) and b) can therefore also be carried out together in a compaction apparatus. If contacting of the lignin-comprising starting material with the pyrolysis catalyst is carried out in the presence of a liquid treatment medium, preference is given to carrying out steps a) and b) at separate times. This makes partial or complete removal of liquid components before densification possible. When a solid lignin-comprising starting material is brought into contact with a solid pyrolysis catalyst, steps a) and b) can be carried out at separate times or partly or completely together.

Contacting of the lignin-comprising starting material with the pyrolysis catalyst is, in a first embodiment, carried out in the presence of a liquid treatment medium. The liquid treatment medium is preferably selected from water, water-miscible organic solvents and mixtures thereof. Suitable water-miscible organic solvents are alcohols such as methanol, ethanol, n-propanol, isopropanol, n-butanol, tert-butanol, diols and polyols, e.g. ethanediol and propanediol, tetrahydrofuran, ketones such as acetone and methyl ethyl ketone and mixtures thereof. Preferred water-miscible organic solvents are methanol and/or ethanol.

The water content of the liquid treatment medium is preferably at least 70% by weight, particularly preferably at least 90% by weight, based on the total weight of the liquid treatment medium. In particular, water is used as liquid treatment medium.

The pyrolysis catalyst is preferably brought into contact as a preparation in the liquid treatment medium with the lignin-comprising starting material. The liquid preparation can be in the form of a solution, colloidal dispersion or dispersion of the catalyst in the treatment medium. Suitable processes for the contacting are, for example, the customary dipping and impregnation processes as are known for catalyst production. The catalyst preparation in the liquid treatment medium is preferably moved past the lignin-comprising starting material during treatment of the latter, e.g. by means of stirring or pumped circulation.

The liquid treatment medium is preferably removed after contacting. This can be carried out using the customary processes, e.g. by evaporation, optionally at elevated temperature and/or under reduced pressure.

In a specific embodiment, contacting of a preparation of the pyrolysis catalyst in the liquid treatment medium with the lignin-comprising starting material is carried out by a modified “incipient wetness method”. Here, excess treatment medium is removed after contacting.

Contacting of the pyrolysis catalyst in a liquid treatment medium with the lignin-comprising starting material is preferably carried out at a temperature of at least 10° C., particularly preferably at least 20° C. The maximum temperature in the treatment is generally not critical, but for practical reasons is preferably below the boiling point of water, e.g. not more than 95° C.

The duration of the contacting of the pyrolysis catalyst in a liquid treatment medium with the lignin-comprising starting material is preferably at least 30 minutes, in particular at least one hour.

Before the densification step, the treated lignin-comprising starting material can additionally be subjected to drying. The temperature during drying is preferably from 30 to 100° C., particularly preferably from 40 to 95° C. Drying can be carried out in apparatuses customary for this purpose, e.g. drying ovens and drying chambers. A gas stream (e.g. a stream of air) can be additionally conveyed past the treated lignin-comprising starting material to aid drying.

In a second embodiment, contacting of the lignin-comprising starting material with the pyrolysis catalyst is carried out in the absence of a liquid treatment medium. Suitable methods of mixing a solid catalyst with the lignin-comprising starting material are known mixing methods, e.g. for producing a powder mixture. Apparatuses known to those skilled in the art, e.g. drum mixers, rotary tubes or other suitable mixers, can be used for this purpose.

Densifying can be carried out in an apparatus customary for this purpose.

Suitable extruders are single-screw machines, intermeshing screw machines or multiple screw extruders, in particular twin-screw extruders. These can be corotating or contrarotating and optionally be equipped with kneading disks. If a solvent has to be vaporized during extrusion, the extruders are generally provided with a vaporization section. Particular preference is given to extruders of the ZKS series from Werner and Pfleiderer.

Tabletting can be carried out using an apparatus customary for this purpose.

Densifying is particularly carried out by compacting. Compacting can be carried out in an apparatus customary for this purpose. Such apparatuses include roller compacters as can be obtained, for example, from Powtec.

The composite obtained can be subjected to a separation according to the particle size in an apparatus suitable for this purpose. Such apparatuses include the sieve devices customary for this purpose. The fraction having a sufficiently large particle size can be separated off as product. The fines in the composite, which have too small a particle size, are generally returned to densification.

The composite obtained can, if desired, be subjected to comminution. Comminution is preferably carried out simultaneously with a separation on a sieving machine.

The composites of the invention and the composites produced by the process of the invention have a particularly advantageous property profile when used in the pyrolysis. These include at least one of the following properties:
1.) high total yield of liquid or liquefiable material of value (which for the purposes of the invention is an organic compound or a composition composed of at least two organic compounds which under normal conditions (20° C., 1013 mbar) are present in liquid form or can be liquefied without decomposition; liquefaction means the transition from the solid state to the liquid state in the sense of melting and not solubilization with addition of a solvent),
2.) high total yield of aromatics based on the starting material,
3.) high proportion of monomeric aromatics in the pyrolysis product,
4.) small proportion of compounds which are relatively nonvolatile under the pyrolysis conditions,
5.) avoidance of tar formation and/or the buildup of adhering layers of carbonaceous material in the reactor space and other parts of the plant,
6.) formation of a (catalyst-comprising) carbonaceous material having a defined particulate structure which aids transport through the plant and/or can readily be recycled.

### Pyrolysis

The above-described composites of the invention and the composites which can be obtained by the above-described process of the invention are advantageous for use in a pyrolysis in which an aromatics composition is obtained as product of value.

The invention therefore also provides a process for preparing an aromatics composition from a lignin-comprising starting material, wherein a composite comprising lignin and at least one acidic pyrolysis catalyst dispersed in the composite is subjected to a pyrolysis. As regards suitable and preferred embodiments of the lignin-comprising starting material, the pyrolysis catalyst and the composite, reference is made to the information given above in its full scope.

For the pyrolysis, the lignin-comprising starting material can be used partly or completely in the form of a composite. The lignin-comprising starting material is preferably used to an extent of at least 50% by weight, preferably at least 70% by weight, in particular at least 90% by weight, in the form of a composite. In a specific embodiment, the lignin-comprising starting material is used completely in the form of a composite for the pyrolysis.

The pyrolysis can be carried out batchwise or continuously. Continuous pyrolysis is preferred.

The pyrolysis is carried out in at least one pyrolysis zone. In a preferred embodiment, the pyrolysis zone comprises at least one fixed bed. The fixed beds can comprise at least one inert fixed bed material. The use of at least one catalytically active fixed bed material is also possible. In the process of the invention, the pyrolysis catalyst is preferably used entirely in the form of a composite.

When the process of the invention is operated using at least one fixed bed as pyrolysis zone, cyclic operation in which a pyrolysis phase is followed by a burning-off phase to remove relatively nonvolatile components from the fixed bed can be advantageous.

In an alternative embodiment, the pyrolysis zone can also be configured, for example, as a rotary tube furnace or fluidized bed. Both stationary and circulating fluidized beds are suitable. In the embodiment of the pyrolysis zone as fluidized bed, a fluidizing gas and a particulate material which is inert under the prevailing conditions as fluidized material are introduced. A particularly suitable inert material is silica sand. Such a fluidized-bed process is described, for example, in U.S. Pat. No. 4,409,416 A.

For the pyrolysis, a feed gas is preferably fed into the pyrolysis zone.

Preferred feed gases comprise at least one gas selected from nitrogen, carbon dioxide, steam, etc., and mixtures of these gases. In addition, the feed gas can comprise at least one further gas which is selected, for example, from hydrogen, methane, carbon monoxide, etc.

In a first preferred embodiment, the pyrolysis is not carried out with addition of hydrogen and/or hydrogen-comprising gases and/or hydrogen-donating compounds. The hydrogen-donating compounds include, in particular, water vapor. In this embodiment, essentially no hydrogenating reaction occurs during the pyrolysis.

In a second preferred embodiment, the pyrolysis is carried out with addition of hydrogen and/or hydrogen-comprising gases and/or hydrogen-donating compounds. This embodiment of the pyrolysis can also be referred to as “hydrocracking”. In this embodiment, the aromatic lignin degradation products formed in the pyrolysis are at least partly transformed by action of hydrogen and/or water vapor so that substituents are replaced by hydrogen and/or compounds comprising a plurality of aromatic rings are cleaved to give compounds having a smaller number of rings. Reactions such as dehydroxylation, dealkoxylation, aromatics cleavage, etc., are also comprised. The substituents replaced by hydrogen are preferably selected from alkyl groups, hydroxy groups, alkoxy groups and aryloxy groups. The feed gas used for hydrocracking preferably has a water vapor content of from 2 to 90% by volume, particularly preferably from 5 to 80% by volume, in particular from 10 to 70% by volume. In the case of steam dealkylation, the hydrogen necessary for the reaction is formed in situ by reaction of water with (mainly organic) components which are either comprised in the feed mixture or are formed during the course of the steam dealkylation. An example which may be mentioned is the formation of hydrogen from methane and water according to the equation CH₄+H₂O→CO+3H₂. If the pyrolysis is carried out with addition of hydrogen and/or hydrogen-comprising gases and/or hydrogen-donating compounds, an aromatics composition comprising low molecular weight aromatic compounds which are preferably selected from benzene and phenolic compounds such as phenol and/or dihydroxybenzenes is formed as target product of the process of the invention. The aromatics composition then has, in particular, smaller proportions of the following components than in the case of a pyrolysis in the presence of hydrogen and/or hydrogen-comprising gases and/or hydrogen-donating compounds: monoalkylated, dialkylated and polyalkylated phenols; alkoxyphenols such as methoxyphenols; polyalkylated benzenes; compounds comprising two or more aromatic rings.

The temperature in the pyrolysis is preferably in the range from 200 to 1500° C., particularly preferably from 350 to 950° C., in particular from 380 to 850° C.

The pressure in the pyrolysis is preferably in the range from 0.5 to 250 bar (absolute), preferably from 1.0 to 40 bar (absolute).

The residence time at the pyrolysis temperature can be from a few seconds to a number of days. In a specific embodiment, the residence time at the pyrolysis temperature is from 0.5 seconds to 5 minutes, especially from 2 seconds to 3 minutes.

Suitable processes for the catalyzed pyrolysis of lignin are, for example, also described in WO 96/09350 (Midwest Research Institute, 1996) or U.S. Pat. No. 4,409,416 (Hydrocarbon Research Institute, 1983), which are hereby incorporated by reference.

In the pyrolysis zone, the lignin is converted into a mixture of components which under the conditions of the pyrolysis are partly present in gaseous form (“pyrolysis gas”) and partly in solid and/or liquid form (e.g. tar-like or as “carbonaceous material”). A discharge is taken from the pyrolysis zone, with it generally being advantageous to discharge the pyrolysis gases and the solid and/or liquid components separately.

The discharge from the pyrolysis zone is preferably subjected to a separation to give the following three streams:
D1) a solid catalyst-comprising fraction,
D2) an aromatics-enriched fraction,
D3) a fraction enriched in by-products which are more volatile than D2).

In general, the discharge from the pyrolysis zone is obtained by taking off a gas discharge either continuously or at intervals during the pyrolysis. This comprises all components which are volatile under the pyrolysis conditions. They include the aromatics composition sought according to the invention and components which are more volatile than this. The solid and/or liquid components which are relatively nonvolatile under the pyrolysis conditions are discharged separately after the pyrolysis is complete. The solid and, if present, liquid components discharged from the pyrolysis zone are, for example, relatively nonvolatile components (carbonaceous material) formed during the pyrolysis and also the pyrolysis catalyst. If at least one solid inert material is used for the pyrolysis, the discharge from the pyrolysis zone can also comprise proportions of the inert material. If the pyrolysis gas still comprises solid and/or liquid components, these can be separated off from the pyrolysis gas by means of a suitable apparatus, e.g. a cyclone.

To obtain the fraction D1), the catalyst is separated from the solid by-products having a high content of carbonaceous material from the pyrolysis reaction. In principle, a number of possibilities are available for the purposes of the process. For example, the by-product comprising carbonaceous material can be thermally utilized and the mineral ash can then be digested. Soluble catalysts can then be separated off using suitable solvents. Suitable solvents are the same solvents used for the treatment of the lignin-comprising starting material with the fresh catalyst. Insoluble catalysts can be freed of all soluble components and be separated off in this way.

Preferred solvents are water, ethanol and methanol; particularly preferred solvents are water or solvents having a water content of greater than 70% by volume, particularly preferably a water content of greater than 90% by volume. Admixing the ash with digesting agents such as alkalis or other useful chemicals known to those skilled in the art and carrying out suitable processes known to those skilled in the art, for example thermal or hydrothermal treatment, is likewise explicitly comprised in the process of the invention.

However, the solid mineral pyrolysis residue comprising the used catalyst can in principle also be converted directly by mechanical methods into a suitable fraction and reacted directly with the lignin-comprising starting material. Here, it can be useful to add a certain amount of fresh catalyst in order to achieve the desired properties. Preference is given to adding only a small amount of fresh catalyst when the catalyst is reused in the process of the invention. Preference is given to fresh catalyst additions of less than 90%, particularly preferably less than 50%, very particularly preferably less than 30%, based on the mass of the reused catalyst material.

It is likewise possible to utilize catalysts or catalyst precursors which, because of their price or other prerequisites, must not be recirculated to the pyrolysis process. Here, particular preference is given to catalysts or catalyst precursors which, after burning off the carbonaceous material, can be recirculated to a leaching process as carried out, for example, in the digestion of wood, e.g. a kraft process.

The pyrolysis product comprises substituted aromatics and/or multiring aromatics. The pyrolysis product can, in addition to aromatics, comprise further components selected from water vapor, inert gas (e.g. nitrogen), nonaromatic hydrocarbons, H₂, CO, CO₂, sulfur-comprising compounds such as H₂S, etc., and mixtures thereof. The nonaromatic hydrocarbons are preferably degradation products such as methane.

The pyrolysis product can be subjected to a separation to give the fractions D2) and D3). Conventional thermal separation processes such as distillation or adsorption can be used for this purpose.

The fraction D2) is the aromatics composition sought as target product and has a high proportion of aromatic compounds having up to 15 aromatic rings. The total content of aromatic compounds having up to 15 aromatic rings is, based on the total weight of the aromatics composition, at least 50% by weight.

The fraction D2) has a high content of single-ring aromatics. The fraction D2) preferably comprises at least 1% by weight, particularly preferably at least 2% by weight, of single-ring aromatics.

The monomeric aromatics comprised in the fraction D2) are preferably selected from benzene, toluene, xylenes, ethylbenzene, phenol, phenol ethers, cresols, xylenols, guaiacols, veratroles, resorcinols, catechols, hydroquinones and further compounds different therefrom.

The fraction D3) comprises components which are, for example, selected from nonaromatic hydrocarbons, especially methane, hydrogen, carbon monoxide, carbon dioxide and mixtures thereof. Depending on the lignin-comprising starting material provided in step a), the stream D3) can comprise further components. When a lignin-comprising starting material from the kraft process is used, these include sulfur-comprising by-products, especially H2S.

The fraction D3) which is depleted in aromatics and enriched in volatile by-products can be passed to various uses. These uses include combustion. If the process of the invention is in the physical vicinity of a pulp process, it can be advantageous to feed fraction D3) into an apparatus of the pulp process. The fraction D3) is particularly preferably fed into the waste liquor combustion (recovery boiler). This embodiment has the advantage that no additional apparatuses for steam or power generation or for flue gas desulfurization in the case of the combustion of fraction D3) are required. In another variant, the combustion of fraction D3) is preceded by a desulfurization, e.g. in the form of a gas scrub to remove hydrogen sulfide, followed by conversion of the H₂S formed into elemental sulfur. The formation of sulfur can be effected by known methods, e.g. the Claus process.

The invention further provides a process for preparing an aromatics composition from a lignin-comprising starting material, wherein:
a) a lignin-comprising starting material is provided,
b) the lignin-comprising starting material is brought into contact with a pyrolysis catalyst and subjected to compaction,
c) the composite obtained in step b) is subjected to pyrolysis in a pyrolysis zone,
d) the discharge from the pyrolysis zone is subjected to separation to give the following three streams:
   D1) a solid catalyst-comprising fraction,
   D2) an aromatics-enriched fraction,
   D3) a fraction enriched in by-products which are more volatile than D2),
e) the fraction D1) obtained in step d) is at least partly recirculated to the pyrolysis zone.

As regards suitable and preferred embodiments of process steps a) to e), reference is made to the information given above in respect of these steps in its full scope.

The pyrolysis product according to the invention or the aromatics-enriched fraction D2 can advantageously be subjected to a dealkylation for further work-up. A dealkylation process has the primary purpose of obtaining a relatively high proportion of low molecular weight aromatic materials of value. For the purposes of the invention, the term “dealkylation” refers to a reaction in which the substituted and/or multiring aromatic compounds comprised in an aromatics composition are at least partly transformed in the presence of hydrogen and/or water vapor so that substituents are replaced by hydrogen and/or compounds comprising a plurality of aromatic rings are cleaved to give compounds having a smaller number of rings. The substituents replaced by hydrogen are here selected from alkyl groups, hydroxy groups, alkoxy groups, aryloxy, etc. For the purposes of the invention, the term “dealkylation” also refers to various reactions which are associated with a decrease in molecular weight, e.g. dehydroxylation, dealkoxylation, aromatics cleavage. Aromatics cleavage here refers to a reaction in which essentially the number of aromatic rings per molecule is reduced without the aromatic rings themselves being destroyed.

In the dealkylation, the aromatic lignin degradation products formed in the pyrolysis are at least partly transformed by action of hydrogen and/or water vapor so that substituents are replaced by hydrogen and/or compounds comprising a plurality of aromatic rings are cleaved to form compounds having a smaller number of rings. As indicated above, the term “dealkylation” also refers to reactions in which no alkyl substituent is replaced by hydrogen, e.g. dehydroxylation, dealkoxylation, aromatics cleavage, etc. The substituents replaced by hydrogen are preferably selected from alkyl groups, hydroxy groups, alkoxy groups and aryloxy groups.

Suitable dealkylation processes comprise hydrodealkylation, steam dealkylation or mixed forms derived therefrom. In the case of a pure hydrodealkylation in the context of the invention, molelcular hydrogen (in pure form or in admixture with other components such as CO) but no water is fed into the dealkylation zone in addition to the pyrolysis product. In the case of a pure steam dealkylation in the context of the invention, water (in pure form or in admixture with other components) but no molecular hydrogen is fed into the dealkylation zone in addition to the pyrolysis product. The dealkylation process can also be configured as a mixed form of hydrodealkylation and steam dealkylation. Both water and molecular hydrogen are then fed into the dealkylation zone in addition to the pyrolysis product. Suitable and preferred process parameters, partly specific to hydrodealkylation and partly to steam dealkylation, are indicated below. Using this information, a person skilled in the art will be able to determine suitable and preferred process parameters for a mixed form of hydrodealkylation and steam dealkylation. The reaction gas used for the dealkylation then preferably has a mixing ratio of H₂ to H₂O in the range from about 0.1:99.9 to 99.9:0.1. An especially suitable mixing ratio of H₂ to H₂O is in the range from about 40:60 to 60:40.

The hydrogen required for the reaction is, in the case of the steam dealkylation, formed in situ by reaction of water with (mainly organic) components which are either comprised in the starting mixture for the steam dealkylation or are formed during the steam dealkylation. An example which may be mentioned here is the formation of hydrogen from methane and water according to the equation CH₄+H₂O→CO+3 H₂.

The temperature in the dealkylation zone is preferably in the range from 400 to 900° C., particularly preferably from 500 to 800° C.

The absolute pressure in the dealkylation zone is preferably in the range from 1 to 100 bar, particularly preferably from 1 to 20 bar, in particular from 1 to 10 bar.

In a first preferred embodiment, the pyrolysis product is subjected to a hydrodealkylation. For this purpose, the reaction is carried out in the presence of hydrogen.

The temperature in the dealkylation zone for the hydrodealkylation is preferably in the range from 500 to 900° C., particularly preferably from 600 to 800° C.

The absolute pressure in the dealkylation zone for the hydrodealkylation is preferably in the range from 1 to 100 bar, particularly preferably from 1 to 20 bar, in particular from 1 to 10 bar.

The ratio of the amount of H₂ used to H₂ (stoichiometric) in the hydrodealkylation is preferably in the range from 0.02 to 50, particularly preferably from 0.2 to 10. Here, H₂ (stoichiometric) is the amount of H₂ which is theoretically required for complete conversion of the aromatics fed into the dealkylation zone into benzene, with the assumption that 1 mol of H₂ reacts per ring substituent.

The residence time in the dealkylation zone for the hydrodealkylation is preferably in the range from 0.1 to 500 s, particularly preferably from 0.5 to 200 s.

In a second preferred embodiment, the pyrolysis product is subjected to a steam dealkylation. For this purpose, the reaction is carried out in the presence of water vapor.

The temperature in the dealkylation zone for the steam dealkylation is preferably in the range from 400 to 800° C., particularly preferably from 475 to 600° C., in particular from 525 to 600° C.

The absolute pressure in the dealkylation zone for the steam dealkylation is preferably in the range from 1 to 100 bar, particularly preferably from 1 to 20 bar, in particular from 1 to 10 bar.

The steam dealkylation can be carried out in the presence or absence of a catalyst. In a specific embodiment, the steam dealkylation is carried out in the absence of a catalyst. A catalyzed process for steam dealkylation is described in WO 2008/148807 A1. This document and the references cited therein in respect of suitable catalysts are hereby fully incorporated by reference. Further information on catalyst types and process steps for steam dealkylation may be found in WO 2007/051852 A1, WO 2007/051851 A1, WO 2007/051855 A2, WO 2007/051856 A1, WO 2008/135581 A1and WO 2008/135582 A1 (EP 2008055585), without being restricted thereby. U.S. Pat. No. 3,775,504 states that a steam dealkylation actually comprises a combination of steam dealkylation and hydrodealkylation, since it is inherent in the system that at least part of the hydrogen produced is immediately reacted again.

In the dealkylation step, at least one low molecular weight aromatic material of value is formed as target product in the process of the invention. The low molecular weight aromatic materials of value are preferably selected from benzene and phenolic compounds such as phenol and/or dihydroxybenzenes.

They have, in particular, smaller proportions of the following components than the pyrolysis discharge before introduction into the dealkylation step: monoalkylated, dialkylated and polyalkylated phenols; alkoxyphenols such as methoxyphenols; polyalkylated benzenes; compounds comprising two or more aromatic rings.

### DESCRIPTION OF THE FIGURES

FIG. 1 shows an apparatus which is suitable for carrying out the process of the invention and is having a fluidized-bed reactor.

FIG. 2 shows a scanning electron micrograph of the composite from example 8 based on a zeolite as pyrolysis catalyst after the pyrolysis.

The invention is illustrated by the following nonlimiting examples.

### EXAMPLES

In all the examples described here, the pyrolysis was carried out in a simple shaft reactor into which the lignin samples were introduced as a fixed bed. The gaseous pyrolysis product formed was discharged continuously, collected in a receiver and the composition was determined gravimetrically after the end of the experiment. After the pyrolysis was complete, the solid contents of the reactor were discharged, the catalyst-comprising carbonaceous material formed was separated from the inert material (metal balls, diameter from 1.5 to 2 mm) and the composition of the carbonaceous material was likewise determined gravimetrically. A gas analysis was not carried out; the proportion of gaseous material was determined by difference. The reactor was heated by means of radiative heating to a temperature of 400° C. over a period of 15 minutes. A GHSV (gas hourly space velocity) of 20 000 h⁻¹, based on the volume of lignin used (amount introduced: 2.5 g), was set by means of the feed gas. The reactor had a diameter of 9 mm. The same proportions by volume of metal balls and lignin were used. When soluble catalysts were used, precompacted lignin was used, this was premixed with the inert metal balls and the catalyst was subsequently applied by the “incipient wetness” method and subsequently dried at 80° C. When solid catalysts were used, all solids were mixed by means of a drum mixer with the aid of ceramic balls as mixing elements and subsequently compacted on a compacter from Powtec. Compacting was carried out using the following parameters:
Roller pressure: 250 bar
Sieve used: 1000 μm
Speed of rotary sieve mill: 100 min⁻¹
Speed of roll mill: 5 min⁻¹
Roller type: grooved
Repetitions: 10

A composite having a particle size of from 500 to 1000 μm, determined by sieve analysis, was used for all pyrolysis experiments.

All yields are based on the dried weight of lignin introduced.

### EXAMPLE 1

Using the above-described general procedure, an untreated lignin (softwood lignin from a LignoBoost process) was subjected to a pyrolysis. When nitrogen was used as feed gas, a proportion of carbonaceous material of 38.5% was obtained. When a mixture of 70% of nitrogen and 30% of water vapor was used as feed gas, a carbonaceous material content of 36% was obtained. When nitrogen was used as feed gas, a biooil yield (aromatics-comprising product) of 38.5% was obtained. The yield of single-ring aromatics was 5.4% by weight, based on the aromatics in the starting material. When a mixture of 70% of nitrogen and 30% of water vapor was used as feed gas, a biooil yield (aromatics-comprising product) of 36% was obtained. The yield of single-ring aromatics was 3.5% by weight, based on the aromatics in the starting material. After removal of the solid reactor contents from the reactor, tar-like and carbonaceous material adhering to the steel balls was observed.

### EXAMPLE 2

Using the above-described general procedure, a lignin (softwood lignin from a LignoBoost process) which had been loaded with 2% by weight of a zeolite (ZSM-5, modulus 40, manufactured by Zeochem) by solid-state mixing and had subsequently been compacted was subjected to a pyrolysis. When nitrogen was used as feed gas, a proportion of carbonaceous material of 38.5% by weight was obtained. When a mixture of 70% of nitrogen and 30% of water vapor was used as feed gas, a content of carbonaceous material of 36% by weight was obtained. When nitrogen was used as feed gas, a biooil yield (aromatics-comprising product) of 53% by weight was obtained. The yield of single-ring aromatics was 3.4% by weight, based on the aromatics in the starting material. When a mixture of 70% of nitrogen and 30% of hydrogen was used as feed gas, a biooil yield (aromatics-comprising product) of 39% by weight was obtained. The yield of single-ring aromatics was 9.5% by weight, based on the aromatics in the starting material. After removal of the solid reactor contents from the reactor, no tar-like and carbonaceous material adhering to the steel balls was observed.

### EXAMPLE 3

Using the above-described general procedure, a lignin (softwood lignin from a LignoBoost process) which had been loaded with 1% by weight of boric acid by aqueous impregnation and had been compacted beforehand was subjected to a pyrolysis. When nitrogen was used as feed gas, a proportion of carbonaceous material of 36% by weight was obtained. When a mixture of 70% of nitrogen and 30% of water vapor was used as feed gas, a proportion of carbonaceous material of 35% by weight was obtained. When nitrogen was used as feed gas, a biooil yield (aromatics-comprising product) of 65% by weight was obtained. When a mixture of 70% of nitrogen and 30% of hydrogen was used as feed gas, a biooil yield (aromatics-comprising product) of 62% by weight, based on dry lignin, was obtained. After removal of the solid reactor contents from the reactor, no tar-like and carbonaceous material adhering to the steel balls was observed.

### EXAMPLE 4

Using the above-described general procedure, a lignin (softwood lignin from a LignoBoost process) which had been loaded with 3.3% by weight of Disperal (from Sasol) by solid-state mixing and had subsequently been compacted was subjected to a pyrolysis. When nitrogen was used as feed gas, a proportion of carbonaceous material of 47% by weight was obtained. When nitrogen was used as feed gas, a biooil yield (aromatics-comprising product) of 40% by weight, based on dry lignin, was obtained. After removal of the solid reactor contents from the reactor, no tar-like and carbonaceous material adhering to the steel balls was observed.

### EXAMPLE 5

Using the above-described general procedure, a lignin (softwood lignin from a LignoBoost process) which had been loaded with 1% by weight of boric acid by aqueous impregnation and had been compacted beforehand was subjected to a pyrolysis. When nitrogen was used as feed gas, a proportion of carbonaceous material of 36% by weight was obtained. When a mixture of 70% of nitrogen and 30% of water vapor was used as feed gas, a proportion of carbonaceous material of 35% by weight was obtained. When nitrogen was used as feed gas, a biooil yield (aromatics-comprising product) of 65% by weight was obtained. When a mixture of 70% of nitrogen and 30% of hydrogen was used as feed gas, a biooil yield (aromatics-comprising product) of 62% by weight, based on dry lignin, was obtained. After removal of the solid reactor contents from the reactor, no tar-like and carbonaceous material adhering to the steel balls was observed.

### EXAMPLE 6

Using the above-described general procedure, a lignin (softwood lignin from a LignoBoost process) which had been loaded with 1% by weight of Ni (based on metal) by aqueous impregnation with nickel acetate and had been compacted beforehand was subjected to a pyrolysis. When nitrogen was used as feed gas, a proportion of carbonaceous material of 48% by weight was obtained. When nitrogen was used as feed gas, a biooil yield (aromatics-comprising product) of 52% by weight, based on dry lignin, was obtained. After removal of the solid reactor contents from the reactor, no tar-like and carbonaceous material adhering to the steel balls was observed.

### EXAMPLE 7

Using the above-described general procedure, a lignin (softwood lignin from a LignoBoost process) which had been loaded with 5% by weight of Co (based on metal) by aqueous impregnation with cobalt acetate and had been compacted beforehand was subjected to a pyrolysis. When nitrogen was used as feed gas, a proportion of carbonaceous material of 64% by weight was obtained. When nitrogen was used as feed gas, a biooil yield (aromatics-comprising product) of 33% by weight, based on dry lignin, was obtained. After removal of the solid reactor contents from the reactor, no tar-like and carbonaceous material adhering to the steel balls was observed.

### EXAMPLE 8

### Pyrolysis in a Circulating Fluidized Bed

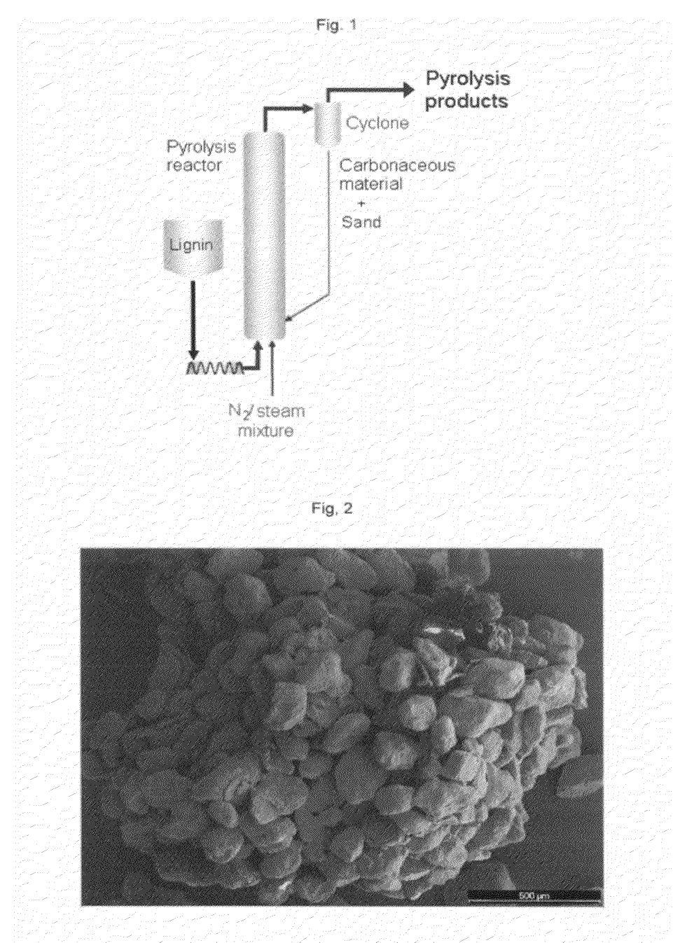

General procedure: a plant as shown in FIG. 1 was used. A continuously introduced, catalyst-modified and compacted lignin was pyrolyzed in a circulating fluidized bed.

The lignin composite was fed in by means of a metering screw. The amount of lignin introduced was determined gravimetrically. The catalyst-modified and compacted lignin had a particle size in the range from 0.5 to 2 mm. The pyrolysis reactor (circulating fluidized bed) had a diameter of 80 mm and a height of 1900 mm. It was electrically heated to 1100° C. Silica sand having an average particle size of 250 μm was used as bed material. After the pyrolysis, the silica sand was recirculated via a cyclone to the reactor. The plant was operated at a pressure of 1.2 bar. An N₂/steam mixture comprising from 40% by volume to 80% by volume of steam was used as fluidizing gas. The gas residence times were in the range from 0.5 to 2 s. The reaction temperature examined was in the range from 500 to 800° C.

In the above-described plant, a lignin which had been modified with a zeolite catalyst and compacted as per example 2 was subjected to a pyrolysis. For this purpose, 2.7 kg of lignin were fed into the reactor over a period of 45 minutes (3.6 kg/h). The reactor temperature was 650° C. The fluidizing gas comprised 19 kg/h of N₂ and 12 kg/h of steam, so that the gas velocity in the reactor was 4 m/s. A proportion of carbonaceous material of 26.4% by weight, based on lignin used, was obtained from the pyrolysis. The biooil yield was 41.1% by weight, based on lignin used. The proportion of single-ring aromatics was 22.6% by weight and the proportion of multiring aromatics was 18.5% by weight, in each case based on lignin used. The uncondensed gases (CO, CO₂, CₙHₘ) had a proportion of 21.1% by weight, based on lignin used.

A significant advantage of the use of a catalyst-modified and compacted lignin according to the invention is that it results in formation of relatively large spherical particles of carbonaceous material which can then be separated off more readily from the silica sand.

1. A composite comprising lignin and at least one pyrolysis catalyst dispersed in the composite.

2. The composite according to claim 1 which comprises lignin in an amount of from 60 to 99% by weight, preferably from 70 to 95% by weight, based on the total weight of the composite.

3. The composite according to either of the preceding claims which comprises the pyrolysis catalyst in an amount of from 0.1 to 20% by weight, preferably from 0.2 to 5% by weight, based on the total weight of the composite.

4. The composite according to any of the preceding claims which has a content of components which are liquid under normal conditions (20° C., 1013 mbar) of not more than 20% by weight, preferably not more than 5% by weight.

5. The composite according to any of the preceding claims, which has an average particle size in the range from 1 to 2000 μm, preferably from 100 to 1000 μm.

6. The composite according to any of the preceding claims which has a bulk density in the range from 0.3 to 0.8 g/l, preferably from 0.4 to 0.7 g/l.

7. A process for producing a composite comprising lignin and at least one pyrolysis catalyst dispersed in the composite, wherein a lignin-comprising starting material is provided and is brought into intimate contact with the pyrolysis catalyst.

8. The process according to claim 7, wherein the material is subjected to densification during and/or after contacting of the lignin-comprising starting material with the pyrolysis catalyst.

9. The process according to claim 8, wherein densification is effected by compacting, extrusion, tabletting or a combination of at least two of these measures.

10. The process according to claim 8, wherein densification is effected by compacting.

11. The process according to any of claims 7 to 10, wherein a lignin-comprising starting material comprising at least 10% by weight, preferably at least 15% by weight, based on the dry mass of the material, of lignin is provided.

12. The process according to any of claims 7 to 11, wherein a lignocellulose material from wood and/or plant fibers is used for providing the lignin-comprising starting material.

13. The process according to any of claims 7 to 12, wherein a lignin-comprising stream from the digestion of a lignocellulose material for producing cellulose (pulp) is used for providing the lignin-comprising starting material.

14. The process according to claim 13, wherein a lignin-comprising stream from the digestion of a lignocellulose material with an alkaline treatment medium, preferably a black liquor, in particular a black liquor from the LignoBoost process, is used for providing the lignin-comprising starting material.

15. The process according to any of claims 7 to 14, wherein the lignin-comprising starting material is subjected to compaction before contacting with the pyrolysis catalyst.

16. The process according to any of claims 7 to 15, wherein the pyrolysis catalyst is selected from boric acid, zeolites, silica, alumina, aluminosilicates, zirconium oxide, titanium dioxide, water-soluble nickel salts, water-soluble cobalt salts and mixtures thereof.

17. The process according to any of claims 7 to 15, wherein the pyrolysis catalyst comprises the catalyst-comprising solid reactor discharge from a pyrolysis in the presence of a composite comprising lignin and at least one pyrolysis catalyst dispersed in the composite.

18. A composite which can be obtained by a process as defined in any of claims 7 to 17.

19. A process for preparing an aromatics composition from a lignin-comprising starting material, wherein a composite comprising lignin and at least one pyrolysis catalyst dispersed in the composite is subjected to a pyrolysis.

20. The process according to claim 19, wherein lignin-comprising starting material is provided entirely in the form of a composite.

21. The process according to either claim 19 or 20, wherein the pyrolysis product is at least partly fed into a dealkylation zone and reacted in the presence of hydrogen and/or water vapor.

22. The process according to either claim 16 or 17, wherein the discharge from the pyrolysis zone is subjected to a separation to give the following three streams:
D1) a solid catalyst-comprising fraction,
D2) an aromatics-enriched fraction,
D3) a fraction enriched in by-products which are more volatile than D2).

23. The process according to claim 18, wherein the fraction D1) is at least partly recirculated to the pyrolysis zone.

24. A process for preparing an aromatics composition from a lignin-comprising starting material, wherein:
a) a lignin-comprising starting material is provided,
b) the lignin-comprising starting material is brought into contact with a pyrolysis catalyst and subjected to compaction,
c) the compacted composite obtained in step b) is subjected to pyrolysis in a pyrolysis zone,
d) the discharge from the pyrolysis zone is subjected to separation to give the following three streams:
D1) a solid catalyst-comprising fraction,
D2) an aromatics-enriched fraction,
D3) a fraction enriched in by-products which are more volatile than D2),
e) the fraction D1) obtained in step d) is at least partly recirculated to the pyrolysis zone.

25. The process according to any of claims 22 to 24, wherein the aromatics-enriched fraction D2) is at least partly fed into a dealkylation zone and reacted in the presence of hydrogen and/or water vapor.
